(19) United States
(12) Patent Application Publication
(10) Pub. No.: US 2008/0139886 A1
(43) Pub. Date: Jun. 12, 2008

(54) Endoscope and bending operation device for endoscope

(75) Inventor: Masanobu Tatsuyama, Hino-shi (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: Olympus Corporation, Tokyo (JP)

(21) Appl. No.: 11/999,700

(22) Filed: Dec. 4, 2007

(30) Foreign Application Priority Data
Dec. 7, 2006 (JP) 2006-331123

(51) Int. Cl. A61B 1/005

(52) U.S. Cl. 600/146

(57) ABSTRACT

An endoscope includes an inserting section having a bending portion, an operating section provided to the inserting section, a bending operation device provided to the operating section, and an operation wire. One end of the operation wire is connected to the bending portion and the other end is connected to the bending operation device. The operation wire is manipulated to bend the bending portion by the bending operation device. The bending operation device includes a handle and a wound body. The handle has a first central axis and is turned around the first central axis. The wound body has a second central axis and is provided to the handle and is fixed to the operation wire. The second central axis of the wound body is provided to be eccentric with respect to the first central axis of the handle.

### CROSS-REFERENCE TO RELATED APPLICATIONS

This application is based upon and claims the benefit of priority from prior Japanese Patent Application No. 2006-331123, filed Dec. 7, 2006, the entire contents of which are incorporated herein by reference.

### BACKGROUND OF THE INVENTION

1. Field of the Invention

The present invention relates to an endoscope having a bending portion and a bending operation device which operates the bending portion, and a bending operation device for an endoscope.

2. Description of the Related Art

As shown in FIGS. 8A to 9C, an endoscope 110 includes an inserting section 112, an operating section 114 and a pair of operation wires 136a and 136b. The inserting section 112 includes a flexible tube portion 126 having the operating section 114 connected to a proximal end side thereof, and a bending portion 124 which is connected with a distal end side of the flexible tube portion 126 and can bend in two directions, i.e., an upper direction and a lower direction from, e.g., a straight state. The pair of operation wires 136a and 136b connects the operating section 114 with the bending portion 124. It is to be noted that two pairs of operation wires are included in case of bending the bending portion 124 in four directions. An example of one pair of operation wires will be explained hereinafter.

When a bending operation knob 172 protruding from a case 152 of the operating section 114 is turned, e.g., counterclockwise from a state depicted in FIGS. 8A and 9A, a pulley 174 fixed to the bending operation knob 172 turns around a central axis C_{b} which also serves as a turning central axis of the bending operation knob 172. Therefore, the operation wires 136a and 136b are respectively fed, pulled in, and moved in directions indicated by arrows in FIG. 8B. Then, the bending portion 124 bends as shown in FIG. 9B. When the bending operation knob 172 is further operated and turned from the state depicted in FIGS. 8B and 9B, the bending portion 124 further bends as shown in FIGS. 8C and 9C.

As disclosed in Jpn. Pat. Appln. KOKAI Publication No. 8-82749, grooves are formed on a pulley which is used for a bending operation in an endoscope. These grooves have trajectories which vary at unequal distances from a turning center of the pulley. Therefore, when winding an operation wire in the grooves of the pulley for one or more turns, the operation wire is smoothly wound on the operation wire corresponding to the first turn from a bottom surface of each groove while preventing the operation wire from rising at a shift position from the first turn to the second turn.

Furthermore, in a structure disclosed in Jpn. Pat. Appln. KOKAI Publication No. 8-82749, when a bending operation knob is turned, an internally provided pulley turns in cooperation with this knob, one of the operation wires is pulled in, and the other is supplied, and a bending portion is bent at an arbitrary angle. When the bending portion is bent to a maximum, a winding radius of the operation wires is minimum, thereby avoiding an increase in a force required for the bending operation.

In FIG. 10, the abscissa represents turning amount (a rotation angle) of the bending operation knob 172, and the ordinate represents distance (change in path length) of the operation wire 136a or 136b from a position at which the operation wire 136a or 136b is separated from the pulley 174 to a proximal end of the bending portion 124.

As shown in FIGS. 8B and 8C, in operations of pulling in and supplying the operation wires 136a and 136b by using the pulley 174 turning around the central axis C_{b} which also serves as the turning central axis of the bending operation knob 172, slack (a surplus amount β₂) of the operation wire 136a or 136b on the supply side is increased when a bending amount of the bending portion 124 is increased (when a supply amount α₂ and a pull amount γ₂ are increased) as shown in FIGS. 9B and 9C and FIG. 10. That is, when the bending amount of the bending portion 124 rises, the supply amount α₂ of the operation wire 136a or 136b greatly deviates from an ideal supply amount (=a supply amount α₀+slack β₀), and the slack (the surplus amount β₂) is gradually increased.

### BRIEF SUMMARY OF THE INVENTION

An endoscope according to an aspect of the present invention includes:

an inserting section including a proximal end and a bending portion which is bent in a plurality of directions;

an operating section provided at the proximal end of the inserting section;

a bending operation device provided to the operating section; and

at least one wire which includes a distal end portion arranged at the bending portion and a proximal end portion arranged at the bending operation device and is manipulated to bend the bending portion by an operation of the bending operation device,

wherein the bending operation device includes:

at least one handle having a first central axis, the handle is turned around the first central axis in a state where the handle is arranged in the operating section; and

at least one wound body having a second central axis, the wound body being provided to the handle and being fixed to the proximal end portion of the operation wire, and the second central axis of the wound body being provided to be eccentric with respect to the first central axis of the handle.

A bending operation device for an endoscope according to an aspect of the present invention, the endoscope including an inserting section having a bending portion and an operation section arranged to the inserting section, the bending operation device arranged at the operating section,

wherein ends of a plurality of operation wires which are used to bend the bending portion of the inserting section of the endoscope being disposed to the bending operation device, supplying and pulling in the operation wires enabling bending the bending portion in a predetermined direction, and

wherein the bending operation device includes:

at least one handle which having a first central axis, the handle being arranged to allow its turning motion around the first central axis in a state where it protrudes toward the outside of the operating section; and

at least one wound body having a second central axis, the wound body being provided to the handle and being fixed to the proximal end portion of the operation wire, and the second central axis of the wound body being provided to be eccentric with respect to the first central axis of the handle.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic perspective view showing an endoscope according to a first embodiment of the present invention;

FIG. 2A is a schematic view showing an outline of an arrangement relationship between an operating section having a bending operation device and an inserting section having a bending portion in the endoscope according to the first embodiment, a pair of operation wires which connect the bending operation device with the bending portion, and a state where the bending portion is straightened;

FIG. 2B is a schematic view showing an outline of an arrangement relationship between the operating section having the bending operation device and the inserting section having the bending portion in the endoscope according to the first embodiment, the pair of operation wires which connect the bending operation device with the bending portion, and a state where a bending operation knob of the bending operation device is turned 90 degrees with respect to the state depicted in FIG. 2A to bend the bending portion;

FIG. 2C is a schematic view showing an outline of an arrangement relationship between the operating section having the bending operation device and the inserting section having the bending portion in the endoscope according to the first embodiment, the pair of operation wires which connect the bending operation device with the bending portion, and a state where the bending operation knob of the bending operation device is turned 180 degrees with respect to the state depicted in FIG. 2A to further bend the bending portion;

FIG. 3A is a schematic view showing the bending operation device in the endoscope according to the first embodiment, the pair of operation wires which connect the bending operation device with the bending portion, and a relationship between the bending operation knob and the operation wires when the bending portion is straightened;

FIG. 3B is a schematic view showing the bending operation device in the endoscope according to the first embodiment, the pair of operation wires which connect the bending operation device with the bending portion, and a relationship between the bending operation knob and the operation wires when the bending operation knob of the bending operation device is turned 90 degrees with respect to the state depicted in FIG. 3A;

FIG. 3C is a schematic view showing the bending operation device in the endoscope according to the first embodiment, the pair of operation wires which connect the bending operation device with the bending portion, and a relationship between the bending operation knob and the operation wires when the bending operation knob of the bending operation device is turned 180 degrees with respect to the state depicted in FIG. 3A;

FIG. 4A is a schematic cross-sectional view showing the bending operation device in the endoscope according to the first embodiment;

FIG. 4B is a schematic view showing a moving state of a pair of pulleys (a trajectory drawn by a central axis Cₚ of the pulleys) with respect to the bending operation knob of the bending operation device in the endoscope according to the first embodiment;

FIG. 5 is a schematic graph showing a change in a path length of each operation wire with respect to a turning angle (a rotation angle) of the bending operation knob of the bending operation device in the endoscope according to the first embodiment;

FIG. 6 is a schematic perspective view showing an endoscope according to a second embodiment of the present invention;

FIG. 7A is a schematic cross-sectional view showing a bending operation device of the endoscope according to the second embodiment;

FIG. 7B is a schematic view showing a moving state of two pairs of pulleys (a trajectory drawn by a central axis Cₚ or Cₚ₁ of the pulleys) with respect to the bending operation knob of the bending operation device in the endoscope according to the second embodiment;

FIG. 8A is a schematic view showing a bending operation device in an endoscope according to conventional technology, a pair of operation wires which connect the bending operation device with a bending portion, and a bending operation knob and the operation wires when the bending portion is straightened;

FIG. 8B is a schematic view showing the bending operation device in the endoscope according to the conventional technology, the pair of operation wires which connect the bending operation device with the bending portion, and a state where the bending operation knob of the bending operation device is turned 90 degrees with respect to the state depicted in FIG. 8A;

FIG. 8C is a schematic view showing the bending operation device of the endoscope according to the conventional technology, the pair of operation wires which connect the bending operation device with the bending portion, and a state where the bending operation knob of the bending operation device is turned 180 degrees with respect to the state depicted in FIG. 8A;

FIG. 9A is a schematic view showing an outline of an arrangement relationship between an operating section having the bending operation device and an inserting section having the bending portion in the endoscope according to the conventional technology, the pair of operation wires which connect the bending operation device with the bending portion, and a state where the bending portion is straightened;

FIG. 9B is a schematic view showing an outline of an arrangement relationship between the operating section having the bending operation device and the inserting section having the bending portion in the endoscope according to the conventional technology, the pair of operation wires which connect the bending operation device with the bending portion, and a state where the bending operation knob of the bending operation device is turned 90 degrees with respect to the state depicted in FIG. 9A to bend the bending portion;

FIG. 9C is a schematic view showing an outline of an arrangement relationship between the operating section having the bending operation device and the inserting section having the bending portion in the endoscope according to the conventional technology, the pair of operation wires which connect the bending operation device with the bending portion, and a state where the bending operation knob of the bending operation device is turned 180 degrees with respect to a state depicted in FIG. 9A to further bend the bending portion; and

FIG. 10 is a schematic graph showing a change in a path length of each operation wire with respect to a turning angle (a rotation angle) of the bending operation knob of the bending operation device in the endoscope according to the conventional technology.

### DETAILED DESCRIPTION OF THE INVENTION

The best mode for carrying out the present invention will now be explained hereinafter with reference to the accompanying drawings.

### First Embodiment

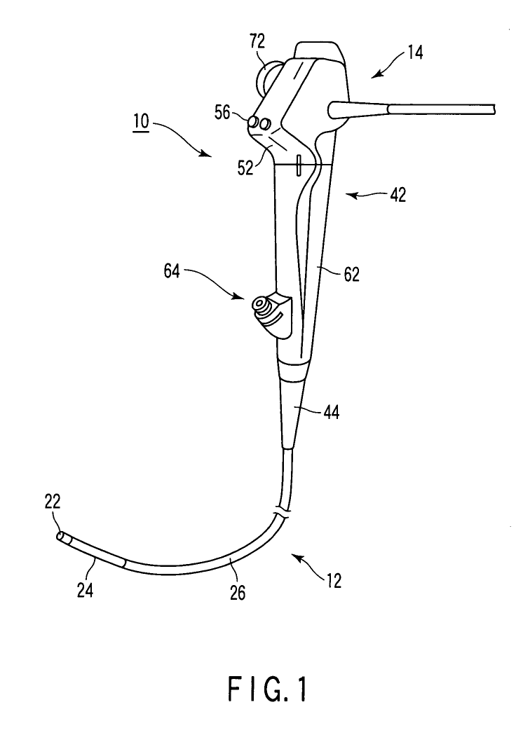

A first embodiment will now be explained with reference to FIGS. 1 to 5.

As shown in FIG. 1, an endoscope 10 includes an elongated inserting section 12 and an operating section 14 arranged at a proximal end of the inserting section 12. The inserting section 12 sequentially includes a distal-end hard portion 22, a bending portion 24, and a flexible tube portion 26 from a distal end side toward a proximal end side.

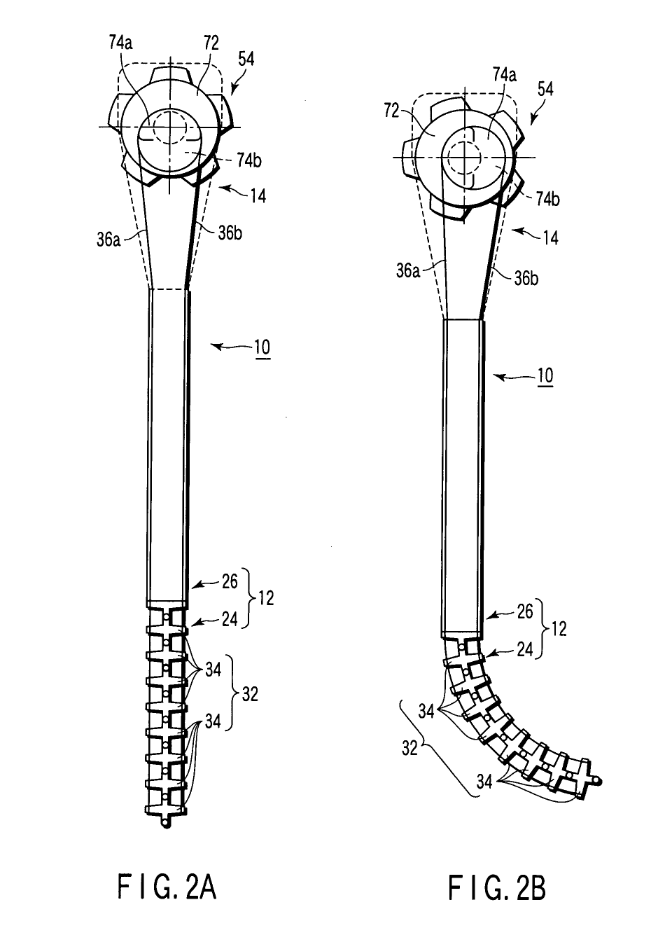

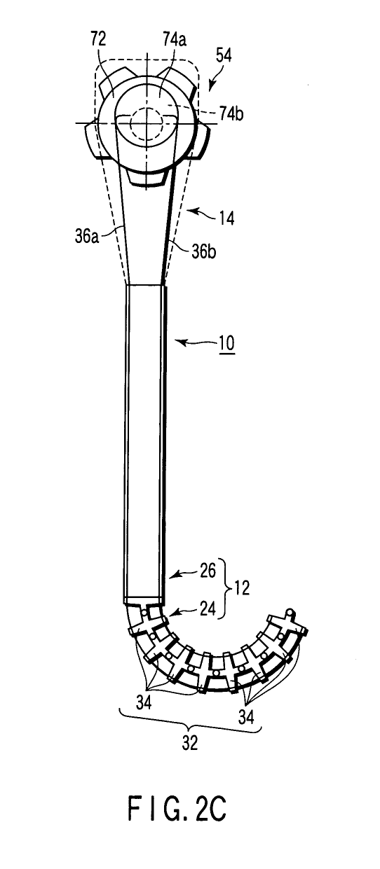

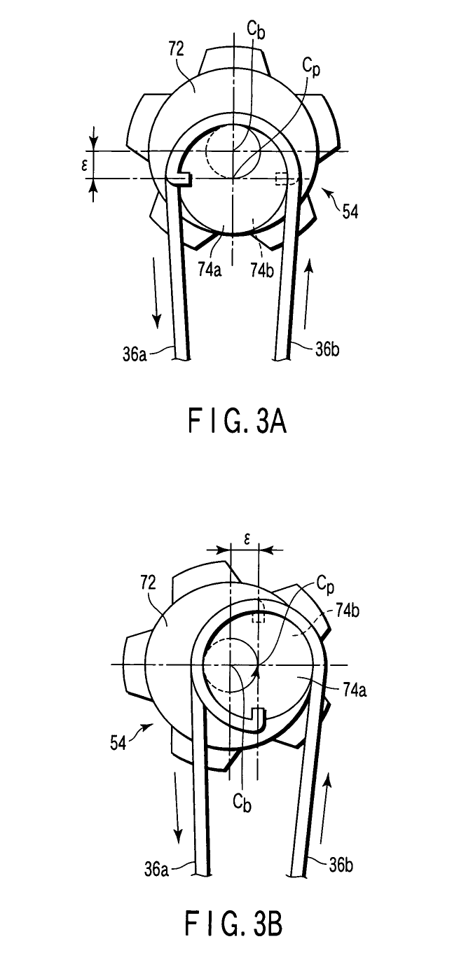

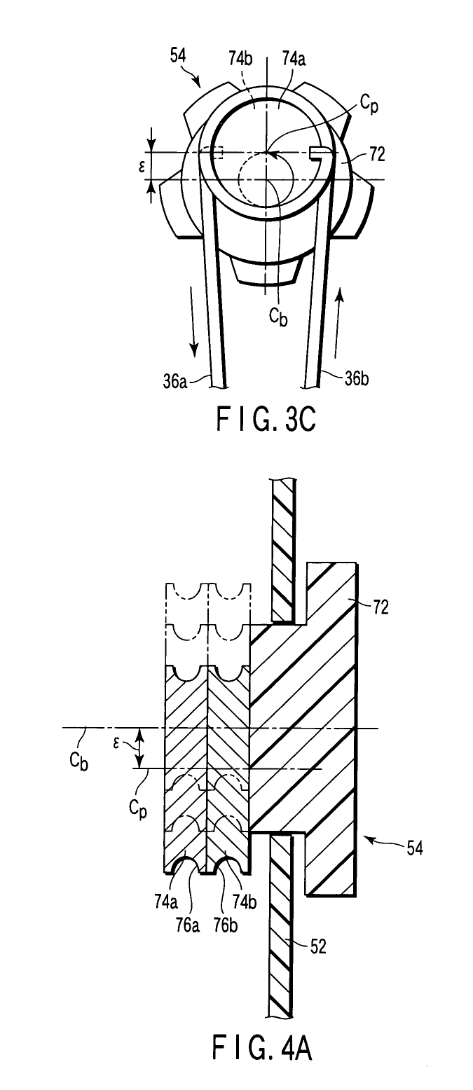

As shown in FIGS. 2A to 2C, in the bending portion 24, a plurality of substantially-ring-shaped bending pieces 34 are aligned along an axial direction of the bending portion 24 and a bending tube 32 supported to allow turning motions of these pieces is arranged. Distal ends of a pair of operation wires 36a and 36b are respectively fixed to the bending piece 34 at the most distal position in this bending tube 32. The operation wires 36a and 36b are guided by wire guide portions (not shown) arranged in the respective bending pieces 34. These wire guide portions are formed at positions where they substantially face central axis lines of the respective bending pieces 34. Therefore, when the pair of operation wires 36a and 36b move in an axial direction of a later-explained bending operation knob 72 by a turning operation of the bending operation knob 72 (when the bending operation knob 72 is turned counterclockwise as shown in FIGS. 3A to 3C, one operation wire 36a is supplied to the distal end side of the inserting section 12 and the other operation wire 36b is pulled in and moved toward the proximal end side of the inserting section 12), the respective bending pieces 34 receive a force. Accordingly, the bending pieces 34 adjacent to each other turn with respect to each other to bend (curve) the bending tube 32, namely, the bending portion 24 bends. In this embodiment, the number of bending directions is two.

As shown in FIG. 1, the operating section 14 includes an operating section main body 42 and a protection hood 44 provided at a proximal end of the inserting section 12. The protection hood 44 is arranged on a lower side of the operating section main body 42 and prevents the flexible tube portion 26 of the inserting section 12 from bending when a large force is applied to the flexible tube portion 26.

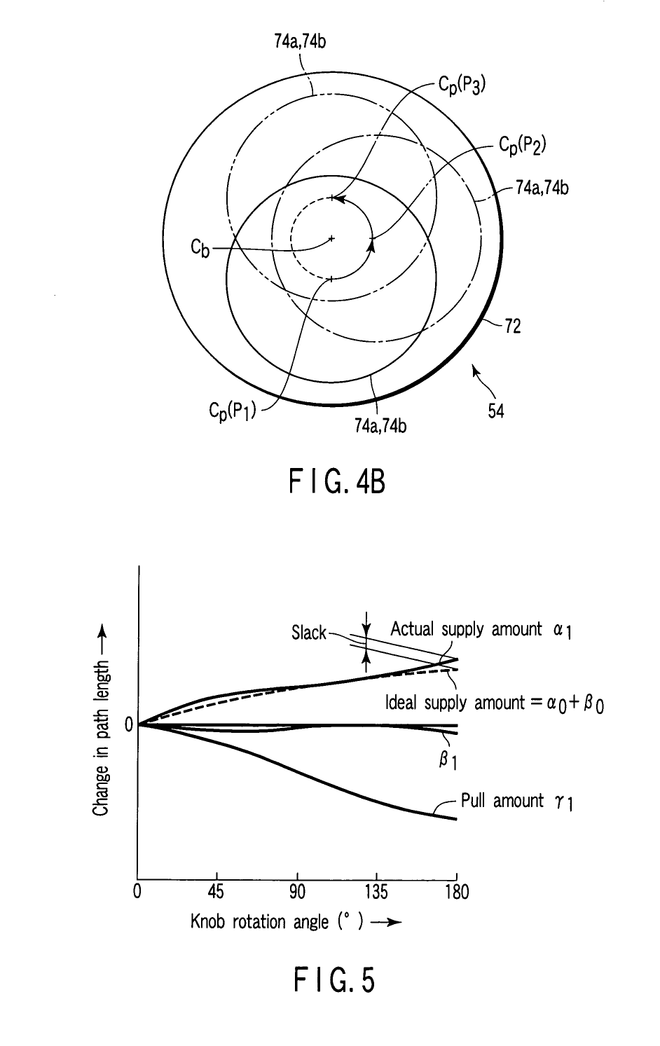

The operating section main body 42 includes a case 52 (see FIG. 4A), a bending operation device 54 arranged in such a manner that a part of a bending operation knob (a handle) 72 protrudes outwards from the case 52, and a plurality of switches 56 which are used to operate an observation optical system or an illumination optical system. A gripping portion 62 which is gripped by an operator or a proximal-end-side opening portion 64 for a forceps channel is formed in the case 52.

As shown in FIG. 4A, the bending operation device 54 includes the bending operation knob (the handle) 72 and a pair of pulleys (wound bodies) 74a and 74b having, e.g., a discoid shape. The bending operation knob 72 partially protrudes from the case 52. The pair of pulleys 74a and 74b are fixed to the bending operation knob 72, and integrally turn with the bending operation knob 72. It is to be noted that a structure of, e.g., a holding substrate which supports an assembled body constituted of the bending operation knob 72 and the pair of pulleys 74a and 74b at a fixed height position with respect to the case 52 in the case 52 is not shown. The pair of pulleys 74a and 74b have the same shape, are integrally fixed, and have the same (common) central axis (a second central axis, a second rotation axis) Cₚ. A part of the bending operation knob 72 protruding from the case 52 of the operating section main body 42 of the operating section 14 is grasped by an operator to be turned. The bending operation knob 72 includes a central axis (a first central axis, a first rotation axis) C_{b} set in a direction substantially perpendicular to an axial direction of the inserting section 12, and is held by the holding substrate (not shown) arranged in the case 52 to allow its turning motion. The pair of pulleys 74a and 74b are coupled and integrally fixed and provided to the bending operation knob 72 by non-illustrated fixing means, e.g., screws or adhesive. It is to be noted that the pair of pulleys 74a and 74b may be formed as an integrated matter when manufacturing the bending operation knob 72, and provided on one end side of the bending operation knob 72. At this time, the central axis Cₚ of the pair of pulleys 74a and 74b deviates on the distal end side of the inserting section 12 from the central axis C_{b} of the bending operation knob 72 by a distance indicated by an eccentric amount ε in a state where the bending portion 24 is extended straight (which is called a neutral state of the bending portion 24).

As shown in FIGS. 3A to 3C, in the bending operation device 54, proximal ends of the operation wires 36a and 36b are respectively fixed to the pulleys 74a and 74b and also wound around in grooves 76a and 76b (see FIG. 4A) of the pulleys 74a and 74b. Moreover, the operation wires 36a and 36b are extended toward the distal end side of the inserting section 12. Therefore, when the curving operation knob 72 is turned around the central axis C_{b} which also serves as a turning central axis of the bending operation knob 72, the pair of pulleys 74a and 74b turn together with the knob 72, and one operation wire 36a is supplied while the other operation wire 36b is pulled in.

It is to be noted that, as shown in FIG. 2A, when the inserting section 12 is straight (the neutral state where the bending portion 24 is straight), the central axis Cₚ of the pulleys 74a and 74b is arranged at a position (a position denoted by reference character P₁ (see FIG. 4B)) where it is approximated to the proximal end of the bending portion 24 (the distal end of the inserting section 12) to a maximum extent as depicted in FIGS. 2A and 3A. At this time, it is assumed that a turning amount (a knob turning angle) of the bending operation knob 72 is 0 degree. Additionally, it is preferable to set positions shown in FIGS. 2C and 3C (−180 degrees or +180 degrees in an opposite direction) as upper limits of the turning amount of the bending operation knob 72. That is, the bending portion 24 bends to a state depicted in FIG. 2C (an opposite state when the knob 72 is turned clockwise).

A function according to this embodiment will now be explained.

As shown in FIG. 2A, in a state where the bending portion 24 is extended straight (a state where the central axis Cₚ of the pair of pulleys 74a and 74b is placed at the position where it is approximated to the proximal end of the bending portion 24 to a maximum extent), the bending operation knob 72 is turned, e.g., 90 degrees counterclockwise to supply or pull in and move the operation wires 36a and 36b in directions indicated by arrows in FIG. 3A. Since the pulleys 74a and 74b are fixed in a state where they deviate from the bending operation knob 72 by a distance corresponding to the eccentric amount ε, the pulleys 74a and 74b rotate one their axis around the central axis C_{b} of the bending operation knob 72 as indicated by alternate long and short dash lines representing outer profile lines of the pulleys 74a and 74b in FIGS. 4A and 4B, and the central axis Cₚ deviating by the eccentric amount ε moves around the central axis C_{b} of the bending operation knob 72. When the bending operation knob 72 is turned, the central axis Cₚ of the pulleys 74a and 74b is arranged to be gradually distanced (a position denoted by reference character P₂) from the proximal end of the bending portion 24 as a bending amount of the bending portion 24 is increased due to revolution in such a deviating state (see FIGS. 2B, 3B, and 4B). Therefore, slack of one operation wire 36a caused by supplying and pulling in the operation wires 36a and 36b is absorbed by movement of the central axis Cₚ of the pulleys 74a and 74b to a distanced side from the proximal end of the bending portion 24.

As shown in FIGS. 2C and 3C, when the bending operation knob 72 is further turned, the central axis Cₚ of the pulleys 74a and 74b is moved to a position (+180 degrees) (a position indicating the outer profile lines of the pulleys 74a and 74b by chain double-dashed lines in FIG. 4B) which is symmetrical to the first position (0 degree) (the neutral state where the bending portion 24 is straight shown in FIG. 2A) with respect to the central axis C_{b} of the bending operation knob 72. That is, the central axis C_{b} of the pulleys 74a and 74b is moved to the most distal side (a position denoted by reference character P₃) with respect to the proximal end of the bending portion 24. Therefore, slack of the one operation wire 36a caused by supplying and pulling in the operation wires 36a and 36b is absorbed by the movement of the central axis Cₚ of the pulleys 74a and 74b to the distal side (the position denoted by reference character P₃) with respect to the proximal end of the bending portion 24.

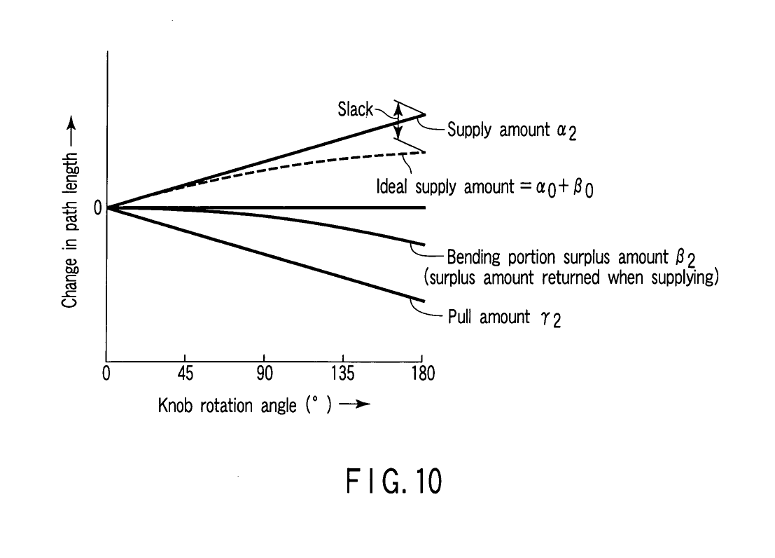

As shown in FIG. 5, using the bending operation device 54 according to the embodiment enables obtaining a supply amount substantially close to an ideal supply amount (=a supply amount α₀+slack β₀) which can avoid occurrence of slack in the operation wire 36a as an actual supply amount α₁ of the one operation wire 36a when the bending operation knob 72 is turned. Here, reference character β₁ denotes a surplus amount (a surplus amount with respect to the supply amount α₁) of the one operation wire 36a supplied to the operating section 14 or the bending portion 24 of the inserting section. The slack (the surplus amount β₁) of the operation wire 36a can be obtained by subtracting the ideal supply amount (=α₀+β₀) from the supply amount α₁. In the embodiment, the surplus amount β₁ of the operation wire 36a supplied in the operating section 14 or the bending portion 24 is greatly reduced as compared with a state of a surplus amount β₂ depicted in FIG. 10.

That is, when the bending operation device 54 according to this embodiment is used, the slack (the surplus amount β₁) on the supply side of the operation wire 36a or 36b is small and a state where the actual supply amount α₁ substantially matches with the ideal supply amount is maintained even if a bending amount of the bending portion 24 is increased (even if the supply amount α₁ and a pull amount γ₁ are increased) as shown in FIGS. 3B and 3C and FIG. 5 in the operation of pulling in and supplying the operation wires 36a and 36b performed by using the pulleys 74a and 74b.

As explained above, according to this embodiment, the following effects can be obtained.

When turning the bending operation knob 72 to bend the bending portion 24, the central axis Cₚ of the pair of pulleys 74a and 74b fixed to the bending operation knob 72 is arranged at the position deviating from the central axis C_{b} of the bending operation knob 72 by the eccentric amount ε (an eccentric position). Further, the pulleys 74a and 74b can be rotated around the central axis C_{b} of the bending operation knob 72 while being rotated on their axis. Therefore, it is possible to change a length between the position of the central axis Cₚ of the pulleys 74a and 74b and the proximal end of the bending portion 24 (a path length) when turning the bending operation knob 72 to bend the bending portion 24. In particular, the length between the position of the central axis Cₚ of the pulleys 74a and 74b and the proximal end of the bending portion 24 can be reduced to a minimum level in the state where the bending portion 24 is straight (the neutral state), and the length between the position (the position denoted by reference character P₃) of the central axis Cₚ of the pulleys 74a and 74b and the proximal end of the bending portion 24 can be increased to a maximum level when the bending portion 24 is bend into the state depicted in FIG. 2C.

Therefore, when turning the bending operation knob 72 to bend the bending portion 24, the pulleys 74a and 74b which are fixed while deviating from the central axis C_{b} of the bending operation knob 72 can be used to bend the bending portion 24 while preventing the slack from occurring in the operation wire 36a or 36b. Accordingly, when restoring the bending portion 24 to the slightly bent state or the neutral state from the greatly bent state, an influence of, e.g., recoil which is caused due to a reduction in the slack of the operation wire 36a and transmitted to the bending operation knob 72 can be avoided as much as possible. Then, when bending the bending portion 24, excellent operability can be obtained in the bending operation knob 72.

Therefore, according to this embodiment, it is possible to provide the endoscope 10 and the bending operation device 54 for the endoscope 10 which can prevent the slack from occurring in the operation wire 36a or 36b to obtain the excellent operability when bending the bending portion 24 and can avoid complication of the structure of the bending portion device 54.

It is to be noted that the example where the inserting section 12 has the flexible tube portion (a flexible tube) 26 has been explained in this embodiment, but a structure where a hard pipe is arranged in place of the flexible tube portion 26 is also preferable.

Furthermore, each of the pulleys 74a and 74b does not have to necessarily have the discoid shape, and an elliptic plate shape and others are also preferable as long as the slack reduced state of the operation wire 36a can be maintained as shown in FIG. 5.

### Second Embodiment

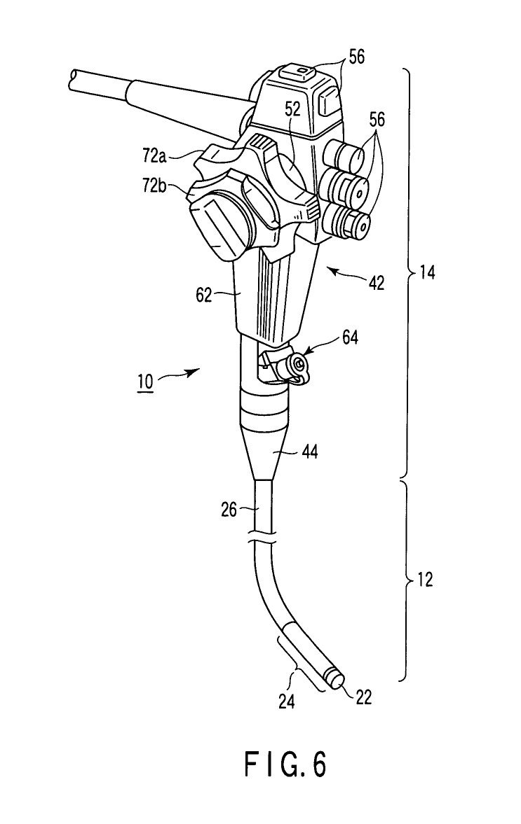

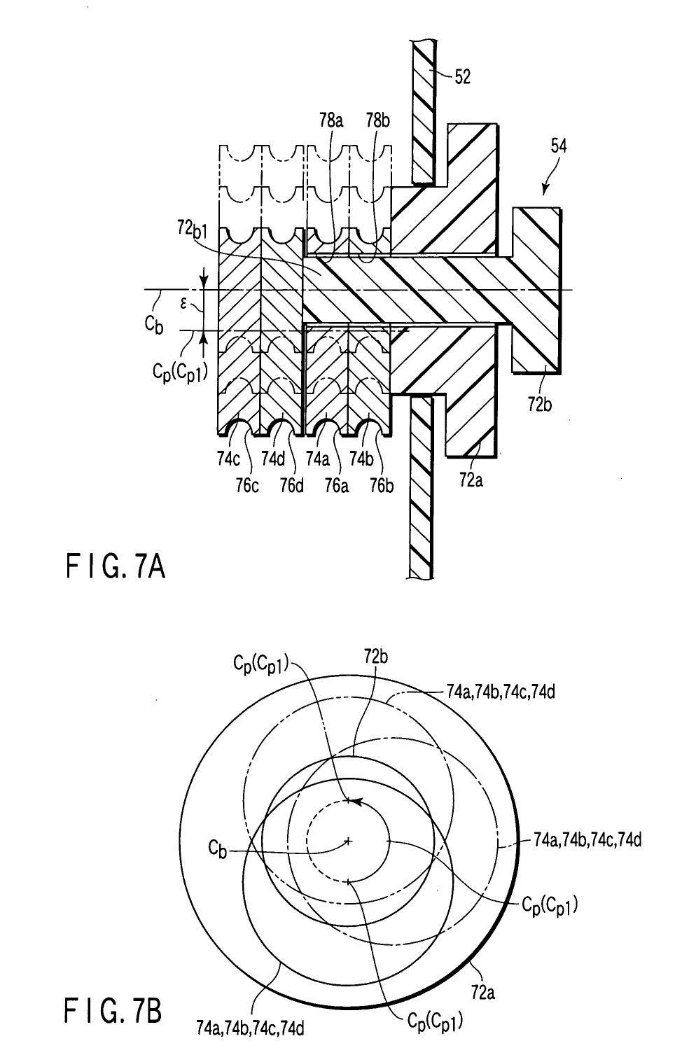

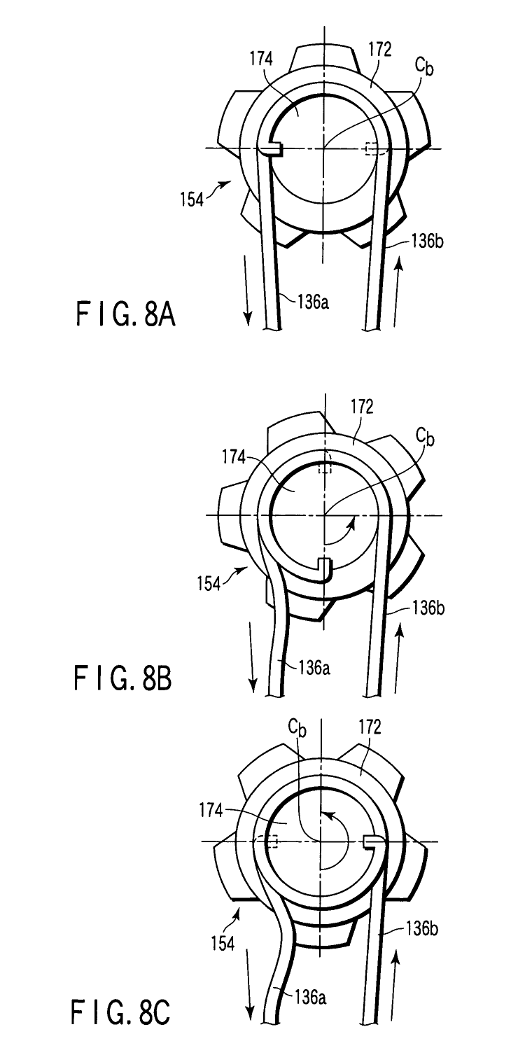

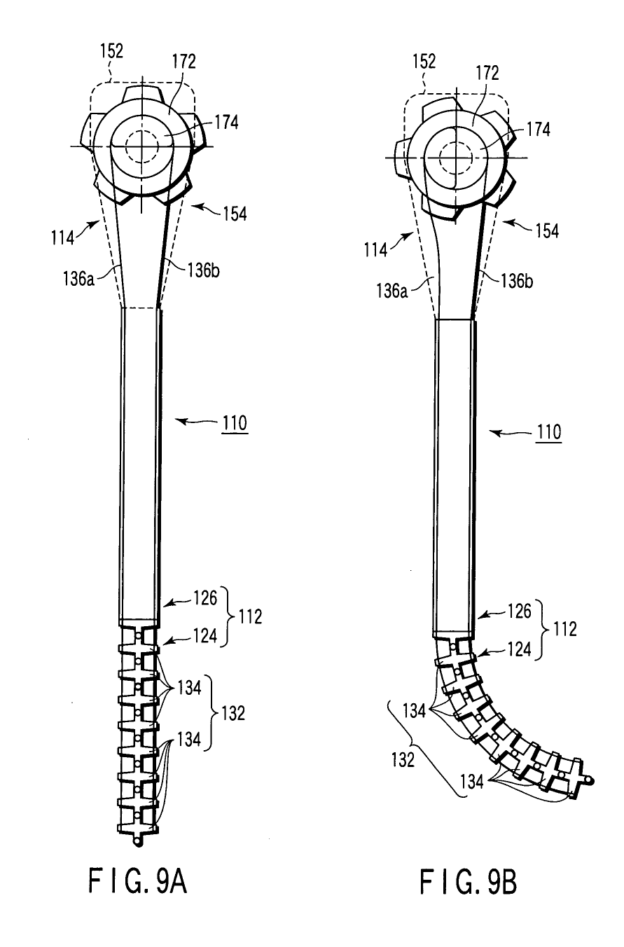

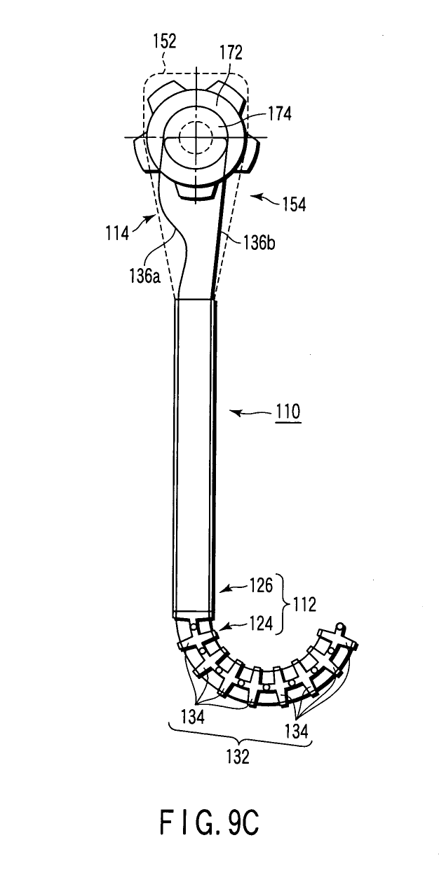

A second embodiment will now be explained with reference to FIGS. 6 to 7B. This embodiment is a modification of the first embodiment, and like reference numerals denote the same members or members having the same functions as those described in the first embodiment, thereby omitting a detailed explanation thereof.

As shown in FIG. 6, according to this embodiment, the two bending directions of the bending portion 24 explained in the first embodiment are expanded to four bending directions.

Arms (protruding portions) which couple non-illustrated neighboring bending pieces to each other are formed at positions of 0 degree and 180 degrees on one end side of each bending piece along a circumferential direction as seen from an axial direction of each bending piece and they are formed at positions of 90 degrees and 270 degrees on the other end side of the same with respect to the bending pieces in a bending portion 24 according to the embodiment. Moreover, of two pairs of operations wires (not shown), one pair of operations wires are guided by wire guide portions formed near the positions of 90 degrees and 270 degrees along the circumferential direction as seen from the axial direction of each bending piece, and the remaining pair of operations wires are guided by wire guide portions formed near the positions of 0 degree and 180 degrees. Therefore, the bending portion 24 can be bent in four directions.

As shown in FIGS. 6 and 7A, an endoscope 10 according to the embodiment is arranged in such a manner that a part of a bending operation knob (a handle) 72 of a bending operation device 54 outwardly protrudes from a case 52 of an operating section main body 42 of an operating section 14. As shown in FIG. 7A, the bending operation device 54 includes a first bending operation knob (a handle) 72a, a second bending operation knob (a handle) 72b, and two pairs of pulleys (wound bodies) 74a, 74b, 74c, and 74d.

The second bending operation knob 72b is arranged to the first bending operation knob 72a. A shaft portion 72_{b1} of the second bending operation knob 72b pierces the first bending operation knob 72a along a central axis C_{b} of the first bending operation knob 72a. The second bending operation knob 72b can turn around the first bending operation knob 72a. These first bending operation knob 72a and second bending operation knob 72b can be independently turned. Additionally, the pair of pulleys 74a and 74b are coupled and integrally fixed to an end portion of the first bending operation knob 72a by non-illustrated fixing means, e.g., screws or an adhesive. The remaining pair of pulleys 74c and 74d are coupled and integrally fixed to an end portion of the shaft portion 72_{b1} of the second bending operation knob 72b by non-illustrated fixing means, e.g., screws or an adhesive. That is, the shaft portion 72_{b1} of the second bending operation knob 72b pierces through holes 78a and 78b of the pair of pulleys 74a and 74b provided at the end portion of the first bending operation knob 72a. The central axis (a first central axis) C_{b} of these through holes 78a and 78b is placed at a position deviating from a central axis (a second central axis) Cₚ of the pulleys 74a and 74b by a distance denoted by a symbol ε.

It is preferable that these pulleys 74a, 74b, 74c, and 74d have a discoid shape. In particular, of the two pairs of pulleys 74a, 74b, 74c, and 74d, one pair of pulleys 74a and 74b are formed into the same shape and fixed to each other, and the remaining (the other) pair of pulleys 74c and 74d are formed into the same shape and fixed to each other. The one pair of pulleys 74a and 74b have the central axis (the second central axis) Cₚ which is common to them. Further, the remaining pair of pulleys 74c and 74d also have a central axis (the second central axis) Cₚ₁ which is common to them. Incidentally, it is also preferable that the two pairs of pulleys 74a, 74b, 74c, and 74d are formed into the same shape. When the bending portion 24 is straight, the central axes Cₚ and Cₚ₁ of the two pairs of pulleys 74a, 74b, 74c, and 74d match with each other, and the two pairs of pulleys 74a, 74b, 74c, and 74d are placed at positions indicated by solid lines in FIG. 7B. That is, the central axes Cₚ and Cₚ₁ of the two pairs of pulleys 74a, 74b, 74c, and 74d are placed at positions close to a proximal end of the bending portion 24 to a maximum extent. Therefore, the central axes Cₚ and Cₚ₁ of the two pairs of the pulleys 74a, 74b, 74c, and 74d deviate from the central axis C_{b} of the first bending operation knob 72a and the second bending operation knob 72b by the eccentric amount ε, respectively.

Furthermore, proximal ends of operation wires 36a, 36b, 36c, and 36d are connected with the respective pulleys 74a, 74b, 74c, and 74d, and wound in grooves 76a, 76b, 76c, and 76d. Moreover, since distal end sides of the operation wires 36a, 36b, 36c, and 36d are arranged at the most distal bending piece through the wire guide portions of the respective bending pieces in the bending portion 24 as explained above, turning the first bending operation knob 72a and the second bending operation knob 72b enables bending the bending portion 24 in four directions.

It is to be noted that other structures, functions, and effects are the same as those described in the first embodiment, thereby omitting an explanation thereof.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an inserting section including a proximal end and a bending portion which is bent in a plurality of directions;
an operating section provided at the proximal end of the inserting section;
a bending operation device provided to the operating section; and
at least one wire which includes a distal end portion arranged at the bending portion and a proximal end portion arranged at the bending operation device and is manipulated to bend the bending portion by an operation of the bending operation device,
wherein the bending operation device includes:
at least one handle having a first central axis, the handle is turned around the first central axis in a state where the handle is arranged in the operating section; and
at least one wound body having a second central axis, the wound body being provided to the handle and being fixed to the proximal end portion of the operation wire, and the second central axis of the wound body being provided to be eccentric with respect to the first central axis of the handle.

2. The endoscope according to claim 1,
wherein the wound body includes discoid body, and
the second central axis of the wound body is rotatable on the second central axis and around the first central axis of the handle so that the second central axis of the wound body is placed at positions close to a proximal end of the bending portion when the bending portion is straight, and the second central axis of the wound body is placed at positions away from the proximal end of the bending portion while rotating on the second central axis of the wound body and bending on the bending portion.

3. The endoscope according to claim 1,
wherein the handle has single handle,
the operation wire has one pair of operation wires,
the wound body has one pair of wound bodies so that the proximal end portion of each of the pair of operation wires is fixed to each wound body, and
the second central axis of each of the pair of wound bodies match with each other.

4. The endoscope according to claim 1,
wherein the handle has two handles,
the operation wire has two pairs of operation wires,
the wound body has two pairs of wound bodies so that the proximal end portion of each of the pair of operation wires is fixed to each wound body, and
the second central axis of each of one pair of wound bodies match with each other and the other remaining pair of wound bodies match with each other.

5. A bending operation device for an endoscope, the endoscope including an inserting section having a bending portion and an operation section arranged to the inserting section, the bending operation device arranged at the operating section,
wherein ends of a plurality of operation wires which are used to bend the bending portion of the inserting section of the endoscope being disposed to the bending operation device, supplying and pulling in the operation wires enabling bending the bending portion in a predetermined direction, and
wherein the bending operation device includes:
at least one handle which having a first central axis, the handle being arranged to allow its turning motion around the first central axis in a state where it protrudes toward the outside of the operating section; and
at least one wound body having a second central axis, the wound body being provided to the handle and being fixed to the proximal end portion of the operation wire, and the second central axis of the wound body being provided to be eccentric with respect to the first central axis of the handle.

6. The device according to claim 5,
wherein the wound body includes discoid body, and
the second central axis of the wound body is rotatable on the second central axis and around the first central axis of the handle so that the second central axis of the wound body is placed at positions close to a proximal end of the bending portion when the bending portion is straight, and the second central axis of the wound body is placed at positions away from the proximal end of the bending portion while rotating on the second central axis of the wound body and bending on the bending portion.

7. The device according to claim 5,
wherein the wound body has one pair of wound bodies so that the proximal end portion of each of the pair of operation wires is fixed to each wound body, and
the second central axis of each of the pair of wound bodies match with each other.

8. The device according to claim 5,
wherein the wound body has two pairs of wound bodies so that the proximal end portion of each of the pair of operation wires is fixed to each wound body, and
the second central axis of each of one pair of wound bodies match with each other and the other remaining pair of wound bodies match with each other.
